(19) United States
(12) Patent Application Publication
(10) Pub. No.: US 2014/0179809 A1
(43) Pub. Date: Jun. 26, 2014

(54) Determination Of Location Of Bacterial Load In The Lungs

(71) Applicant: Avisa Pharma Inc., Albuquerque, NM (US)

(72) Inventor: Elizabeth A. Perkett, Albuquerque, NM (US)

(73) Assignee: Avisa Pharma Inc., Albuquerque, NM (US)

(21) Appl. No.: 14/104,338

(22) Filed: Dec. 12, 2013

Related U.S. Application Data
(60) Provisional application No. 61/736,239, filed on Dec. 12, 2012.

(51) Int. Cl. C12Q 1/04

(52) U.S. Cl. CPC C12Q 1/04
USPC 514/789; 435/34

(57) ABSTRACT

The present invention is direct to methods of determining the location of a bacterial load in the lungs of a subject.

### CROSS REFERENCE TO RELATED APPLICATIONS

This application claims the benefit of U.S. Provisional Application No. 61/736,239, filed Dec. 12, 2012, the entirety of which is incorporated by reference herein.

### TECHNICAL FIELD

The invention is directed to methods of detecting the presence and location of bacterial load in the lungs of a subject.

### BACKGROUND

Bacteria are naturally present in the lungs and if the bacterial load remains low, these bacteria will not adversely affect normal respiratory function. The presence of bacteria is called colonization, rather than infection. When the bacterial load increases in the upper airway, it may still be colonization and is generally not life threatening, but increased colonization may precede infection so measures may be started to decrease colonization before severe infection occurs. Increased colonization can be treated using non-aggressive methods, for example, by increasing airway clearance or by administering oral or inhaled broad-spectrum antibiotics.

Deeper into the lower airways of lung, bacteria are less common An increase in bacterial load in the lower airways is often associated with infection—with the bacteria being more invasive. Increased bacterial load in the lower airways (“lower respiratory tract”) can be life-threatening, resulting in infections such as pneumonia. Increased bacterial load in the lower airways requires more aggressive treatment, for example, broad spectrum intravenous antibiotics.

A rapid test that can determine whether an increased bacterial load is in the upper or lower respiratory tract would be helpful in determining an appropriate treatment.

### SUMMARY

The present invention is directed to methods for determining the presence or absence and location of a bacterial load in the respiratory system of a subject comprising: administering to the subject, an effective amount of a ¹³C-isotopically-labeled compound that produces ¹³CO₂ upon bacterial metabolism; collecting a plurality of samples of exhaled breath from the subject; at least one of said samples comprising breath from the upper respiratory tract of the subject; and at least one of said samples comprising breath from the lower respiratory tract of the subject; conducting at least some of the samples to a sample chamber of a detection apparatus; evaluating the isotopic ratio of ¹³CO₂ to ¹²CO₂ present in each of the at least some samples; and relating the isotopic ratios thus ascertained to the location in the respiratory system from which said samples conducted to the sample chamber were collected.

### BRIEF DESCRIPTION OF THE DRAWINGS

FIG. 1 is a plan view of an exemplary laser absorbance device for use in accordance with some embodiments of this invention.

FIG. 2 illustrates a preferred jump scanning regime.

### DETAILED DESCRIPTION OF ILLUSTRATIVE EMBODIMENTS

Methods of determining whether a subject has a bacterial lung infection have been previously described. These methods include, for example, administering to the subject a ¹³C-isotopically-labeled compound that produces ¹³CO₂ upon bacterial metabolism. Samples of exhaled breath are then collected and analyzed to determine the isotopic ratio of ¹³CO₂ to ¹²CO₂ present in the samples. An increase in the isotopic ratio of ¹³CO₂ to ¹²CO₂ in the exhaled breath samples, as compared to a control sample, is indicative of a bacterial lung infection. See, e.g., U.S. Provisional Application No. 61/715,992 and U.S. Pat. No. 7,771,857.

Those prior methods, however, did not provide any information about the location of the infection in the lung. Those methods also could not differentiate between colonization and infection. The present invention is directed to methods for determining the location of increased bacterial load in the lung. Any bacteria that can convert the ¹³C-isotopically-labeled compounds of the invention into ¹³CO₂ can be detected using the methods of the invention. Examples of such bacteria include Pseudomonas aeruginosa, Staphylococcus aureus, Mycobacterium tuberculosis, Acenitobacter baumannii, Klebsiella pneumonia, Francisella tularenis, Proteus mirabilis, and Aspergillus species.

An entire exhaled breath sample from a subject will include air from both the upper and lower airways. Air from the lower airways has a higher concentration of carbon dioxide than air from the upper airways. It is generally understood that air from the lower airways of a healthy adult has a pressure of CO₂ of about 40 mm Hg. John F. Murray, The Normal Lung, 2d ed. W.B. Saunders Company, Philadelphia 1986, page 184. Air from the upper airways generally has a CO₂ pressure of less than 1 mm Hg. Id. As such, one skilled in the art can determine whether an exhaled breath sample came from the upper or lower airways by determining the CO₂ concentration. Samples having a higher CO₂ concentration come from lower airways, whereas samples having lower CO₂ concentration come from upper airways.

“Capnography” is known in the art as the monitoring of the concentration or partial pressure of carbon dioxide in respiratory gases. Apparatuses and methods for performing that monitoring are known by those of skill in the art. See, e.g., U.S. Pat. No. 3,830,630; U.S. Pat. No. 7,122,154; and Schubert J. K., et al. CO₂-controlled sampling of alveolar gas in mechanically ventilated patients. J. Appl. Physiol. (1985). 2001 February; 90(2):486-92.

Active pressure sensing can also be used to determine from where in the lung an exhaled breath originated. See, e.g., WO 2008/060165; U.S. Pat. No. 7,547,285. Alternatively, passive pressure sensing can be used to channel and isolate samples from the upper and lower respiratory tract. See, e.g., Bio-VOC™ Breath Sampler (Markes International Limited, United Kingdom); U.S. Pat. No. 3,734,692; WO 1994/018885; WO 2003/049595; and WO 2004/032727.

Determination of the origin of an exhaled breath sample can also be achieved by measuring the temperature of the breath sample. See, e.g., U.S. Pat. No. 4,248,245. Alternatively, exhaled breath is monitored using transthoracic impedance methods, which are known in the art.

It is also understood in the art that in an entire exhaled breath, samples exhaled first will be from the upper airways, while samples exhaled later in time will be from the lower airways. As a result, the skilled person can correlate the location of the exhaled breath sample to the point in time during the entire exhalation at which the sample was collected.

The methods of the invention include administering to the subject, an effective amount of a ¹³C-isotopically-labeled compound that produces ¹³CO₂ upon bacterial metabolism. Exemplary examples of such compounds include isotopically labeled urea, isotopically labeled glycine, isotopically labeled citrulline, or a mixture thereof. Administration of the ¹³C-isotopically-labeled compound can be achieved by any known means. Preferred methods of administration include inhalation and ingestion. Administration via injection, i.e., intramuscular, subcutaneous, peritoneal, and intradermal injection, is also within the scope of the invention.

In some embodiments of the invention, the ¹³C-isotopically-labeled compound is administered to a specific area of the respiratory tract. For example, in certain embodiments, the ¹³C-isotopically-labeled compound is delivered to the lower regions of the lungs, i.e., alveolar regions. In some embodiments, the ¹³C-isotopically-labeled compound is delivered to the upper regions of the lungs. In other embodiments, the ¹³C-isotopically-labeled compound is delivered to the bronchial areas of the lungs. In yet other embodiments, the ¹³C-isotopically-labeled compound is delivered to peripheral areas of the lungs. Methods and devices for targeting delivery of compounds to specific areas of the respiratory tract are known in the art. See, e.g., U.S. Pat. No. 8,534,277.

Within the scope of the invention, one or more exhaled breath samples from the subject can be collected before administration of the ¹³C-isotopically-labeled compound. Such samples can be used as control samples in the methods of the invention. Alternatively, the control samples can include the isotopic ratio of ¹³CO₂ to ¹²CO₂ present in exhaled breath of a population that has not been administered the ¹³C-isotopically-labeled compound.

Following a suitable time period after administration of the ¹³C-isotopically-labeled compound, a plurality of samples of exhaled breath are collected from the subject. A “suitable time period” refers to the length of time required for the compound to be converted to carbon dioxide by a bacteria. Preferably, the samples are collected after no more than 40-70 minutes following administration.

In some embodiments, the time required by the subject to complete a substantially complete exhalation can be evaluated. The subject's breathing patterns can also be evaluated. Those evaluations can be used to, for example, determine preselected time periods during exhalation for sampling.

Samples can be collected in any vessel suitable for containing samples of exhaled breath, for example, a bag or vial. Samples may also be directly exhaled into the device by using a suitable mouthpiece. Samples can also be directed exhaled into the sample chamber of a detection apparatus device by being collected using a nasal cannula from a suitable port on other respiratory equipment, for example, a ventilator.

At least one of the exhaled breath samples will be from the upper respiratory tract and at least one of the exhaled breath samples will be from the lower respiratory tract of the subject. The skilled person can identify the origin of the exhaled breath sample by determining the relative carbon dioxide concentration of the sample. A higher carbon dioxide concentration is indicative of the sample originating from the lower airways. A lower carbon dioxide concentration is indicative of the sample originating from the upper airways.

Alternatively, the skilled person can correlate the origin of the exhaled breath sample to the point in time of sample collection. A sample collected at or near the beginning of the entire exhalation will have originated from the upper airways. A sample collected at or near the end of the entire exhalation will have originated from the lower airways.

The origin of the exhaled breath can also be determined using any of the methods known in the art, such as, for example, capnography, active pressure sending, passive pressure sensing, temperature sensing, and transthoracic impedance.

The samples are analyzed to determine the isotopic ratio of ¹³CO₂ to ¹²CO₂ in the samples. Preferably, at least a majority of the exhaled breaths, and most preferably every exhaled breath, is sampled for a given time period or until the determination of the level of activity has reached a preset accuracy. By correlating the isotopic ratio of the sample to the sample origin, the skilled person can determine whether there is an increase in bacterial load and whether that increase is in the upper or lower airways.

The sample is conducted to a sample chamber of a detection apparatus. A laser light source of the detection apparatus is actuated to emit one or more of the wavelength pairs 2054.37 and 2052.42; 2054.96 and 2051.67; or 2760.53 and 2760.08 nanometers. The laser light thus actuated is directed through the sample in the sample chamber to impinge upon a detector for such wavelengths. The isotopic ratio of ¹³CO₂ to ¹²CO₂ present in the sample can then be ascertained.

A graph or curve may be generated showing the ratio of ¹³CO₂ to ¹²CO₂ in the breath of the tested subject as a function of time. A curve showing an increase in the ratio of ¹³CO₂ to ¹²CO₂ over time is evidence of the existence of a bacterial infection.

The concentrations or amounts (ratio) of ¹³CO₂ to ¹²CO₂ is compared to a standard concentration (ratio) of ¹³CO₂ to ¹²CO₂ in a healthy subject and a curve is conveniently generated. From the curve, the presence or absence of increased bacterial load may be determined or diagnosed directly. Other methods for comparing the output ratio to ratios expected from healthy subjects may also be employed.

In exemplary embodiments, a curve may be fitted to these measured concentrations and is then analyzed, preferably by determining the rate of rise of the curve. Such an analysis (rising rate) indicates the level of activity of bacterial load in the subject, which can be used to diagnose the presence and extent of bacterial load in the subject. This same approach may be used, with modification, to determine the effectiveness of therapy and the prognosis for inhibition and/or a cure of infection or colonization.

Within the scope of the invention are methods of detecting the presence or absence of a bacterial load in a subject by comparing the isotopic ratio of ¹³CO₂ to ¹²CO₂ in the exhaled breath samples obtained after administration of the ¹³C-isotopically labeled compound to the isotopic ratio of ¹³CO₂ to ¹²CO₂ in an exhaled breath sample obtained from the subject prior to the administration of the ¹³C-isotopically labeled compound.

Within the scope of the invention, an increase in the ratio of ¹³CO₂ to ¹²CO₂ in the exhaled breath samples obtained after inhalation of the ¹³C-isotopically labeled compound to the isotopic ratio of ¹³CO₂ to ¹²CO₂ in the exhaled breath sample obtained from the subject prior to the inhalation of the ¹³C-isotopically labeled compound indicates the presence a bacterial load. If that sample originated from the upper airways, colonization is likely present in the upper airways. If that sample originated from the lower airways, infection is likely present in the lower airways.

Once it is determined whether the increased bacterial load is in the upper or lower airways, appropriate therapies can be initiated. For example, if the increased bacterial load is in the upper airways, increased airway clearance in the respiratory tract of the subject can be initiated. Oral or inhaled antibiotics, or other there suitable therapeutic agents, can also be administered.

If the increased bacterial load is in the lower airways, more aggressive treatment can be considered. Such treatments may include, for example, administering therapeutic agents. Such agents include, for example, antibiotics such as broad spectrum, intravenous antibiotics.

Detection apparatuses useful in the present invention will include a sample chamber, into which breath samples can be conducted. These devices will also include a laser light source actuated to emit one or more of the wavelength pairs 2054.37 and 2052.42; 2054.96 and 2051.67; or 2760.53 and 2760.08 nanometers. These devices will also include a detector for detection of one or more of the wavelength pairs.

The detection apparatuses useful in the present invention can include small, extremely low power, near infrared diode lasers to attain field portable, battery operated δ¹³CO₂ measurement instruments with high degrees of accuracy and sensitivity. These devices and the methodologies which employ them may be used to determine δ¹³CO₂ in exhaled breath samples of subjects having, or suspected of having, a bacterial colonization or infection.

Preferred detection apparatuses will analyze carbon isotope ratios in exhaled carbon dioxide samples without being adversely affected by temperature changes. The accuracy and precision of measuring carbon dioxide isotope ratios can be affected by changes in the ground state population of carbon dioxide. The origins of the isotopic differences in samples may be diverse and are not the subject of the present invention. Rather, it is recognized that ascertaining the value of the isotopic ratio is inherently important and commercially useful.

Optical absorption spectroscopy is based on the well-known Beer-Lambert Law. Gas concentrations are determined by measuring the change in the laser beam intensity, I₀, due to optical absorption of the beam by a sample of the gas. If a sample cell is used for the analysis, such that the path length of the beam and inherent characteristics of the measuring device are constant, absorbance measurements allow calculation of the gas number density, n, or gas concentration.

Gas phase diode laser absorption measurements interrogate individual absorption lines of gas molecules. These absorption lines correspond to the transition of the gas molecule, e.g. carbon dioxide, from a ground energy state to a higher excited energy state by absorption of a photon of light. The lines are typically quite narrow at reduced sample gas pressure thereby permitting selective detection of a gas in the presence of other background gases such as water vapor. The isotopes of CO₂ have distinct absorption lines that occur at shifted wavelengths with respect to each other due to the mass difference between ¹²C and ¹³C.

Absorbance measurements are affected by the gas temperature and the magnitude of this temperature sensitivity varies depending on absorption line selection and the total ground state energy of the optical transition. A collection of molecules at room temperature is distributed over many discrete molecular energy states that vary in total energy according to how fast the molecules rotate and vibrate. That is, the ground state molecular population is distributed about discrete rotational and vibrational energy states according to a Boltzmann distribution.

A temperature dependence of Δδ¹³CO₂ can affect the long term stability and sensitivity of diode laser based isotopic measurements of carbon dioxide. [references 2-6] ¹³CO₂ and ¹²CO₂ absorption lines with near equal ground state energies can be useful in attaining relative temperature insensitivity for isotopic ratio measurements.

Vertical cavity surface emitting lasers (VCSELs) have been shown to attain scan ranges of 10 to 15 cm⁻¹. These have been used to give rise to rugged, high precision field instruments as exemplified by a laser hygrometer manufactured by Southwest Sciences, Inc and a handheld methane leak detector manufactured by the Southern Cross Company. Accordingly, for certain apparatuses for use in the invention, VCSELs can be used that may be scanned over the desired spectral wavelengths, at a useful scan rate in the context of an overall testing apparatus as to give rise to some or all of the desired benefits of the present invention. In some embodiments, the VCSEL devices are caused to scan in the kilohertz scan rate or greater over approximately 10 cm⁻¹ ranges.

Suitable laser sources may also be formed from a plurality, usually a pair of laser emitters. Such emitters may be fabricated to emit at one of the preferred wavelengths of a wavelength pair. VCSEL devices useful in the invention may be ordered from Vertilas GmbH of Germany and can also be made by other sources of laser emitters.

Pairs of ¹³CO₂ and ¹²CO₂ spectral lines have been identified, each pair of which has near zero ground state energy difference, a line separation less than 12 cm⁻¹, and is substantially free of water interference. It is now been discovered that these pairs of lines are highly useful in the ascertainment of ¹³CO₂/¹²CO² isotopic ratios in gas samples. The temperature dependence of measurement using these pairs is desirably low.

The spectral line pairs as follows are highly useful in making carbon dioxide isotopic absorption measurements using VCSELs in gas cells in analyzing exhaled breath samples:

| ¹²CO₂ wavelength (nm) | ¹³CO₂ wavelength (nm) |
|---|---|
| 2054.37 | 2052.42 |
| 2054.96 | 2051.67 |
| 2760.53 | 2760.08 |

It will be appreciated that the wavelengths identified in the foregoing line pairs are nominal and that some variation from the listed values may be useful. In this regard, it will be understood that useful wavelengths will be those which are sufficiently close to the recited values as to provide one or more of the benefits of the present invention. Thus, such wavelengths will confer either improved accuracy, improved temperature stability or another of the desirable properties set forth herein to the measurement of CO₂ isotopic ratios. In general, preferred wavelengths will be within 0.5 of a nanometer of the recited values.

In addition to the laser light source operating at the desired wavelengths, the apparatuses useful with the present invention include a sample container for holding the gas sample, which container is configured to provide a relatively long light path through the sample by way of mirrors. One or more signal detectors are included as is control circuitry for controlling the laser and for collecting and manipulating the output signal from the detector or detectors. Other equipment to facilitate sample collection, sample preparation, data interpretation and display and other things may also be included in systems and kits provided by this invention. All such components are preferably sufficiently rugged as to permit the deployment of the devices outside of a laboratory and even in a hand held context.

The present apparatuses are also useful in a system or kit. Components of the system or kit may include sample collection containers, such as gas tight bags, preferably ones featuring injection ports, syringes, and other items which facilitate sample collection and transfer to the sample chamber of the apparatus. Such sample collection elements may assume different configurations depending upon the source of the gas to be sampled. Thus, the same may, for example, be useful for collecting breath of a subject, such as when sampling headspace gases from the stomach of a subject.

Portable devices and systems are known having a general arrangement of elements suitable for us in some of the embodiments of the present invention. For example, the '96 Hawk hand-held methane leak detector system sold by Southern Cross Corp. provides sample container, mirror assemblies, power supply, sample handling and other components which may be adapted for use in the invention. Such systems, however, are not otherwise amenable for such use. Thus, the provision of diode laser sources which are capable of scanning the requisite spectral line pairs with effective frequency, stability and accuracy must be accomplished. Likewise, detectors for sensing optical absorption in the selected line pairs with needed accuracy as well as data collection, storage, manipulation and display or reporting devices and/or software is needed.

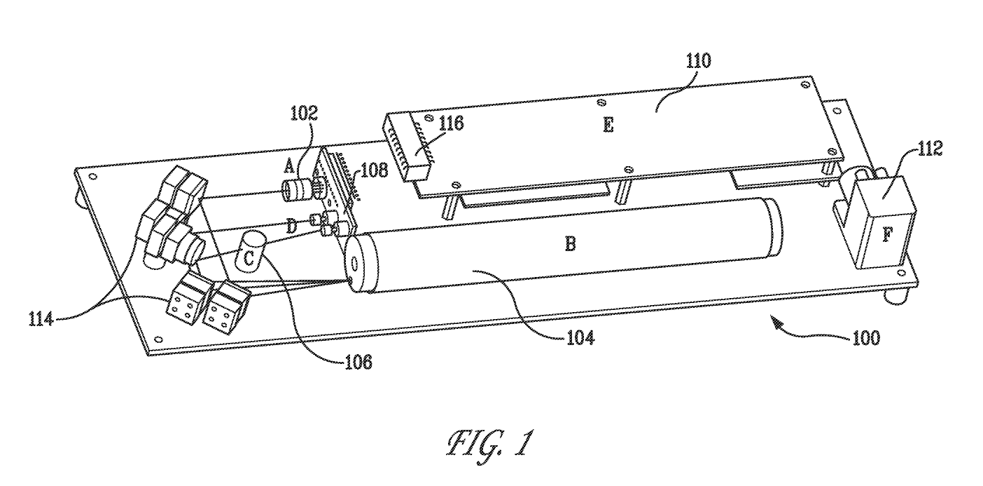

FIG. 1 depicts certain aspects of one device that can be used with the presenting invention. A CO₂ optical absorption measurement device is depicted 100, which comprises a diode laser source, mirrors 114, and gas sample chamber 104. Taken together, these form an optical path in conjunction with preferred reflective surfaces inside the sample chamber, not shown. The optical path, which is effectively many times longer than the physical length of the chamber, permits the enhanced absorption of laser light by gas samples in the chamber. One or more gas pumps, 112 are conveniently included to transport gas sample into and out of the sample chamber which may, likewise, be provided with one or more pressure gauges. Preferably, a reference gas chamber, 106 is also employed together with mirrors, 114 for directing laser light through the reference gas chamber 106. The light paths through the sample and reference chambers are directed to one or more detectors, 108 for assessing the intensity of laser light. Processor or processors in control module, 110 determine the amount of absorption of incident laser light by the sample in the sample chamber, by reference to the reference sample in the reference chamber. This determination may be performed by routine software of firmware, either on board the device or external to it. Preferably, electrical connections, 116 are provided enabling either signals or processed data from the device to be ported to external display or data collection and manipulation devices. In accordance with certain preferred embodiments, some or all of the elements making up apparatuses and systems of the invention and the functions they perform are operated under the control of a controller. Such controller, which may be on board the instrument or external to it, may be a general purpose digital computational device or a special purpose digital or digital—analog device or devices. Control by the controller may be of, for example, power supplies for the laser, detector, gas sample pump, processors and other components.

In operation, a gas sample suspected of containing carbon dioxide is placed into the sample chamber of the devices of the invention. The laser light source or sources is then caused to transit the sample chamber, preferably via a recurring pathway so as to increase the overall path length and improve the measurement sensitivity. The light source is then directed to one or more sensors and the sensor readings interpreted to give rise to a value for wavelength absorption by the sample. The methodologies for making this determination are well known in the art, and include, for example, direct absorption spectroscopy, wavelength modulation spectroscopy, cavity ringdown spectroscopy, and other alternatives By comparing the absorption of light having each of the chosen pair of wavelengths, values for the carbon 12 and carbon 13 isotopes in the carbon dioxide sample become known. Perforce, their ratio may be calculated. For some of the preferred embodiments of the invention, a reference gas sample is provided and the same irradiated, detected and the signal interpreted. The data thus obtained is used to standardize the data arising from irradiation of the sample chamber.

The mechanics of the apparatus including the supply of power to the laser light source or sources, to the detectors and to any data storage, presentation and manipulation elements is preferably under the control of a controller, whether digital or analog. A digital computer may also or in addition be used. Such computer may be on board or connected via a control interface.

It is preferred that determination of light absorption in accordance with the present invention be accomplished by wavelength modulation spectroscopy (WMS). While WMS has been used previously for δ¹³CO₂ measurements [17], it has never been performed for the line pairs that have now been determined to be used for isotopic ratios determinations in carbon dioxide.

WMS is preferred to direct absorption spectroscopy for use in the present invention, although direct measurement may be used if desired. For direct absorbance measurements the laser current is ramped so that the wavelength output is repeatedly scanned across a gas absorption line and the spectra generated are co-averaged. Analysis of direct absorption spectra involves detecting small changes on a large detector signal. For very low concentration changes this is problematic. To perform WMS, a small high-frequency sinusoidal modulation is superimposed on the diode laser current ramp. This current modulation produces a modulation of the laser wavelength at the same high frequency. Absorption by the target gas converts the wavelength modulation to an amplitude modulation of the laser intensity incident on the detector, adding AC components to the detector photocurrent. The detector photocurrent is demodulated at twice the modulation frequency, 2f detection. This selectively amplifies only the AC components (a zero background measurement) and shifts the measurement from near DC to higher frequencies where laser noise is reduced. Spectral noise is greatly reduced by performing signal detection at frequencies (>10 kHz) high enough to avoid fluctuations in the laser output power, laser excess (1/f) noise. In carefully optimized laboratory setups, WMS has measured absorbances as low as 1×10⁻⁷, which is near the detector noise limit. However, in compact field instrumentation, background artifacts typically limit the minimum detectable absorbance αₘᵢₙ to 1×10⁻⁵ s^{-1/2}. The value for αₘᵢₙ can be improved by longer time averaging of the 2f signal with the improvement scaling as t^{1/2} for periods of 100 to 300 seconds.

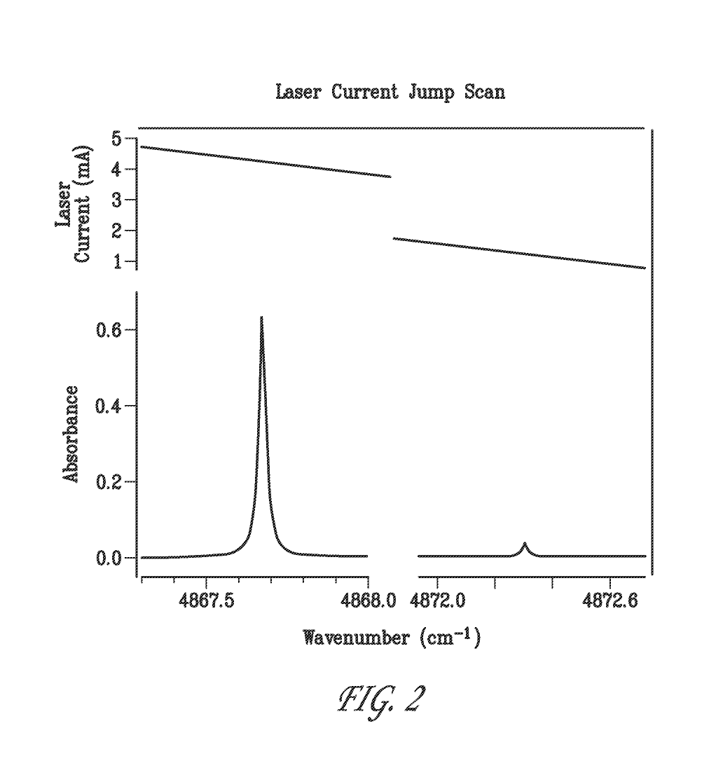

The ¹³CO₂ and ¹²CO₂ absorption line pairs described herein give rise to relatively temperature insensitive δ¹³CO₂ isotopic ratio determinations in gas samples are separated by several absorption lines that do not need to be measured. Instead of continuously scanning the laser wavelength between the two peaks of interest in each pair, the electronics is caused to operate the laser in a jump scan fashion. This is illustrated in FIG. 2. The laser current scan is programmed to have a discontinuity that will rapidly change the wavelength. The first few data points after the jump are preferably not used, as the laser wavelength may not be stable immediately after the current jump. VCSELs used in the present invention may be operated in this way even with four current jumps in order to measure five different absorption lines simultaneously with no undue reduction in sensitivity.

Compositions for oral administration or inhalation, i.e., pulmonary, administration are as otherwise described herein. Oral compositions include powders or granules, suspensions or solutions in water or non-aqueous media, sachets, capsules or tablets. Thickeners, diluents, flavorings, dispersing aids, emulsifiers or binders may be desirable. Compositions for pulmonary administration include a pharmaceutically acceptable carrier, additive or excipient, as well as a propellant and optionally, a solvent and/or a dispersant to facilitate pulmonary delivery to the subject.

Sterile compositions for injection can be prepared according to methods known in the art.

While the present invention has been set forth with reference to numerous embodiments and alternatives, the present specification is not to be taken to be limiting. The invention is solely measured by its claims.

### REFERENCES

1. Bell, G. D., et al., 14C-urea breath analysis, a non-invasive test for Campylobacter pylori in the stomach. Lancet, 1987. 1: p. 1367-1368.

2. Chelboun, J. and P. Kocna, Isotope selective nondispersive infrared spectrometry can compete with isotope ratio mass spectrometry in cumulative 13CO2 breath tests: assessment of accuracy. Kin. Biochem. Metab., 2005. 13(34): p. 92-97.

3. Castrillo, A., et al., Measuring the 13C/12C isotope ratio in atmospheric CO2 by means of laser absorption spectrometry: a new perspective based on a 2.05-μm diode laser. Isotopes in Environmental and Health Studies, 2006. 42(1): p. 47-56.

4. Gagliardi, G., et al., High-precision determination of the 13CO2/12CO2 isotope ratio using a portable 2.008-μm diode-laser spectrometer. Appl. Phys. B, 2003. 77: p. 119-124.

5. Horner, G., et al., Isotope selective analysis of CO2 with tunable diode laser (TDL) spectroscopy in the NIR. Analyst, 2004. 129: p. 772-778.

6. Wahl, E. H., et al., Applications of cavity ring-down spectroscopy to high precision isotope ratio measurement of 13C 12C in carbon dioxide. Isotopes in Environmental and Health Studies, 2006. 42: p. 21-35.

7. Hovde, D. C., et al. Trace Gas Detection Using Vertical Cavity Surface Emitting Lasers. in Optical Remote Sensing for Environmental and Process Monitoring. 1995. San Francisco, Calif.

8. U.S. Pat. No. 6,800,855

9. U.S. Pat. No. 5,929,442

All references cited herein are incorporated by reference in their entireties.

What is claimed:

1. A method for determining the presence or absence and location of a bacterial load in the respiratory system of a subject comprising:
a. administering to the subject, an effective amount of a ¹³C-isotopically-labeled compound that produces ¹³CO₂ upon bacterial metabolism;
b. collecting a plurality of samples of exhaled breath from the subject;
i. at least one of said samples comprising breath from the upper respiratory tract of the subject; and
ii. at least one of said samples comprising breath from the lower respiratory tract of the subject;
c. conducting at least some of the samples to a sample chamber of a detection apparatus;
d. evaluating the isotopic ratio of ¹³CO₂ to ¹²CO₂ present in each of the at least some samples; and
e. relating the isotopic ratios thus ascertained to the location in the respiratory system from which said samples conducted to the sample chamber were collected.

2. The method of claim 1 wherein the isotopic ratios of at least some of the samples conducted to the sample chamber are determinative of the presence or absence of the bacterial load at the locations in the respiratory system from which the respective samples were collected.

3. The method of claim 1 further comprising
a. actuating a laser light source of the detection apparatus to emit one or more of the wavelength pairs 2054.37 and 2052.42; 2054.96 and 2051.67; or 2760.53 and 2760.08 nanometers; and
b. directing the laser light thus actuated through the sample in the sample chamber to impinge upon a detector for such wavelengths.

4. The method of claim 1 further comprising comparing the isotopic ratio of at least one sample conducted to the sample chamber with the isotopic ratio of a control sample to effect said determination.

5. The method of claim 4, wherein the control sample comprises at least one sample of exhaled breath from the subject prior to administration of the ¹³C-isotopically-labeled compound.

6. The method of claim 4, wherein the control sample includes the isotopic ratio of ¹³CO₂ to ¹²CO₂ present in exhaled breath of a population that has not been administered the ¹³C-isotopically-labeled compound.

7. The method of claim 1 wherein the location of said samples is determined by collecting each of said samples during a preselected time period during exhalation by the subject.

8. The method of claim 7 wherein the time period for collection of the plurality of samples is determined following an evaluation of the breathing pattern of the subject.

9. The method of claim 8 wherein said evaluation comprises measurement of the time required by the subject to complete a substantially complete exhalation.

10. The method of claim 1 wherein the location in the respiratory system of at least some of said samples conducted to the sample chamber is determined by ascertaining the total carbon dioxide level in the breath of the subject in the samples.

11. The method of claim 1, wherein the bacterial load is in the lung.

12. The method of claim 1, wherein the ¹³C-isotopically-labeled compound is administered by inhalation.

13. The method of claim 1, wherein the ¹³C-isotopically-labeled compound is administered by ingestion.

14. The method of claim 1, wherein the ¹³C-isotopically-labeled compound is administered by injection.

15. The method of claim 1, the determination being of the presence of a bacterial load of Pseudomonas aeruginosa, Staphylococcus aureus, Mycobacterium tuberculosis, Acenitobacter baumannii, Klebsiella pneumonia, Francisella tularenis, Proteus mirabilis, or Aspergillus species.

16. The method of claim 3, wherein the apparatus further comprises a processor for interpreting or presenting the signals received by the detector.

17. The method of claim 3, wherein the apparatus further comprises one or more of power supply, gas pump, pressure gauge, signal processor, and reference gas chamber.

18. The method of claim 3, wherein the laser light source of the apparatus scans the pair of wavelengths using wavelength modulation spectroscopy.

19. The method of claim 3, wherein the wavelength pair is 2054.37 and 2052.42 nanometers.

20. The method of claim 3, wherein the wavelength pair is 2051.67 and 2054.96 nanometers.

21. The method of claim 3, wherein the wavelength pair is 2760.53 and 2760.08 nanometers.

22. The method of claim 3, wherein the laser light source of the apparatus comprises a pair of laser emitters.

23. The method of claim 3, wherein the laser light source of the apparatus is a vertical cavity surface emitting laser.

24. The method of claim 1, wherein the ¹³C-isotopically-labeled compound is isotopically labeled urea, isotopically labeled glycine, isotopically labeled citrulline, or mixture thereof.

25. The method of claim 1 wherein the isotopically labeled compound is ¹³C-labeled urea.

26. The method of claim 1 wherein the isotopically labeled compound is a mixture of ¹³C-labeled urea and ¹³C-labeled glycine.

27. The method of claim 1 further comprising comparing the isotopic ratio of ¹³CO₂ to ¹²CO₂ in the evaluated exhaled breath samples obtained after administration of the ¹³C-isotopically labeled compound to the isotopic ratio of ¹³CO₂ to ¹²CO₂ in at least one exhaled breath sample obtained from the subject prior to the administration of the ¹³C-isotopically labeled compound.

28. The method of claim 1, wherein an increase in the ratio of ¹³CO₂ to ¹²CO₂ in at least some of the samples conducted to the sample chamber before inhalation of the ¹³C-isotopically labeled compound to the isotopic ratio of ¹³CO₂ to ¹²CO₂ in the at least one exhaled breath sample obtained from the subject prior to the inhalation of the ¹³C-isotopically labeled compound indicates the presence of a bacterial load in a lung of the subject.

29. The method of claim 1, wherein an increase in the isotopic ratio of ¹³CO₂ to ¹²CO₂ before inhalation of the ¹³C-isotopically labeled compound to the isotopic ratio of ¹³CO₂ to ¹²CO₂ in the at least one exhaled breath sample from the upper respiratory tract obtained from the subject indicates the presence of bacterial colonization in the upper respiratory tract of the subject.

30. The method of claim 1, wherein an increase in the isotopic ratio of ¹³CO₂ to ¹²CO₂ before inhalation of the ¹³C-isotopically labeled compound to the isotopic ratio of ¹³CO₂ to ¹²CO₂ in the at least one exhaled breath sample from the lower respiratory tract obtained from the subject indicates the presence of a bacterial infection in the lower respiratory tract of the subject.

31. The method of claim 29, further comprising the step of increasing airway clearance in the respiratory tract of the subject.

32. The method of claim 29 or 30, further comprising the step of administering to the subject a therapeutic agent for reducing said colonization or infection.
